(19) United States
(12) Patent Application Publication
(10) Pub. No.: US 2024/0335589 A1
(43) Pub. Date: Oct. 10, 2024

(54) METHODS AND APPARATUS FOR DECELLULARIZATION OF BIOLOGICAL TISSUES AND ORGANS

(71) Applicant: Agency for Science, Technology and Research, Connexis North Tower (SG)

(72) Inventors: Deepak Choudhury, Centros (SG); Jia Sheng Lee, Centros (SG); Ahmad Amirul, Centros (SG); May Win Naing, Innovis (SG)

(21) Appl. No.: 18/294,106

(22) PCT Filed: Aug. 4, 2022
(86) PCT No.: PCT/SG2022/050558

§ 371 Date: Jan. 31, 2024

(30) Foreign Application Priority Data
Aug. 4, 2021 (SG) 10202108515R

(51) Int. Cl. A61L 27/36

(52) U.S. Cl. CPC A61L 27/3633; A61L 27/3683; A61L 2430/40

(57) ABSTRACT

The invention relates to a method for decellularization of tissue samples to produce decellularized extracellular matrix (dECM) by performing one or more wash cycles in a reactor using a reagent after at least partially submerging the tissue sample in the reagent, wherein the reactor comprising a tissue chamber in fluid communication with a reactor chamber. The method further comprising the steps of agitating the tissue sample and/or the reagent such that the tissue sample circulates in the reagent while retaining the tissue sample in a tissue chamber or a decellularization reactor, monitoring at least one parameter of the reagent and/or at least one parameter of the tissue sample, and adjusting one or more wash cycles based on said monitoring. The invention further relates to a decellularization reactor and a decellularization system for decellularizing a tissue sample.

### TECHNICAL FIELD

The present disclosure relates to methods and apparatus for decellularization, for example for decellularization of animal organs, animal tissues, human organs, human tissues, plant tissues, fungi (mushroom) tissues, algae tissues and/or waste valorized samples from any of the above.

### BACKGROUND

The process of obtaining decellularized extracellular matrix (dECM), also known as decellularization, is a relatively new lab process with its technologies and devices being largely non-specific, undefined and only available in small, lab scales. Decellularization removes cellular and nuclear components from tissues while preserving the ultrastructure of ECM components; collagen, elastin, glycosaminoglycans (GAGs) and so on in animal and human samples, and cellulose, pectin, lignin and so on in plant samples.

One application of decellularization is in organ regeneration; a fully decellularized organ could be recellularized with relevant cells and potentially grown and mature for a transplantation in future. Another application relates to 3D bioprinting technology, which is used in tissue engineering to additively and precisely build specific 3D complex living tissue, while depositing cells, biomaterials and biomolecules. dECM can be used in the bioinks to provide cells with the natural microenvironment niche, and complex cues to direct cellular processes, which regular hydrogels are unable to do. Furthermore, dECM provides a scaffold that has already been adapted to the housing and adhering of cells.

A large majority of research in decellularization techniques has been conducted using perfusion of whole animal organs, with the goal of recellularization to obtain functioning organs that can potentially reduce the need for allogeneic transplants. These involve controlling the flow of detergents and reagents perfusing through the organs and engineered unique protocols that use oscillating pressure or osmotic shock to obtain decellularized organs.

Other than whole organ decellularization, techniques that use tissue samples or smaller denominations of animal organs (like minced samples) have been explored, albeit less. Most of them follow the decellularization processes using agitation methods for the production of bioink. This method of decellularization and bioink formation has been adopted to produce skeletal tissue and muscular tissue. Beyond decellularization of animal organs and tissue samples, there is minimal progress in decellularization devices and processes for tissue samples from other sources such as humans, animals, plants, fungi, or algae. The decellularized plants, fungi, algae could be used to construct scaffolds, coatings or ingredients of plant-based meat alternatives or cultured/cultivated/lab-grown meat products.

To date, there has been significantly more research in decellularization of whole organs for recellularization than techniques for smaller animal organs. Meanwhile, there has been scarce development of decellularization techniques of smaller tissues with standardized shapes and sizes. Though these techniques allow for the recellularization and potential reconstruction of whole organs someday, these are not essentially useful for 3D bioprinting or other technologies that require the use of dECM hydrogels as the base material.

Known methods of decellularization to obtain these dECM commonly use improvised lab-ware or commercial devices that are limited in applications. These set-ups and methods have low production scales and lack the ability to track the progress of decellularization. The consequence is an inefficient process which can be costly while underutilizing resources and tissue samples.

Further advancements in immersion and agitation techniques are needed to ensure uniform and consistent decellularization throughout samples, and across batches. Current methods employ non-standardized steps, while using crude and non-specialized equipment. Hence, current methods tend to yield inconsistently decellularized tissue samples in non-specific shapes. Though these are sufficient in creating hydrogels in the lab, they are inadequate in generating scalable amounts of dECM.

Production of scalable amounts of dECM requires repeatability and reproducibility of the decellularization process, in order to generate products with consistent quality and characteristics. Additionally, efficient handling procedures, universality, monitoring and real-time optimizing capabilities will further improve the quality, consistency, and scalability of decellularization.

### SUMMARY

The present disclosure relates to a method for decellularizing a tissue sample to produce decellularized extracellular matrix (dECM) material, the method comprising:
placing the tissue sample in a tissue chamber that is in fluid communication with a reactor chamber;
performing one or more wash cycles each comprising: at least partially filling the reactor chamber with a reagent such that the tissue sample is at least partially submerged in the reagent; and agitating the tissue sample and/or the reagent such that the tissue sample circulates in the reagent, while retaining the tissue sample in the tissue chamber;
monitoring at least one parameter of the reagent and/or at least one parameter of the tissue sample; and
adjusting one or more wash cycles based on said monitoring.

The present disclosure also relates to a decellularization reactor for decellularizing a tissue sample to produce decellularized extracellular matrix (dECM) material, the decellularization reactor comprising:
a reactor chamber;
a tissue chamber that is in fluid communication with the reactor chamber, the tissue chamber comprising a base and one or more sidewalls extending from the base, at least one of said one or more sidewalls comprising a plurality of apertures for passage of fluid between the tissue chamber and the reactor chamber while retaining the tissue sample within the tissue chamber;
a port in fluid communication with the reactor chamber and the tissue chamber for introducing reagent into the chambers in use; and
an agitation system for agitating, in use, the reagent and/or the tissue sample such that the tissue sample circulates in the reagent in the tissue chamber.

The present disclosure further relates to a decellularization system comprising:
a decellularization reactor as disclosed herein;
a reagent delivery system connectable to the port of the decellularization reactor; and
a controller that is operably connectable to the reagent delivery system and the agitation system, the controller being configured to cause the decellularization reactor to perform one or more wash cycles each comprising: at least partially filling, by the reagent delivery system, the reactor chamber with reagent such that the tissue sample is at least partially submerged in the reagent; and agitating, by the agitation system, the tissue sample and/or the reagent such that the tissue sample circulates within the reagent, while retaining the tissue sample in the tissue chamber.

### BRIEF DESCRIPTION OF THE DRAWINGS

Some embodiments of a method, system, and apparatus for decellularization, in accordance with present teachings, will now be described, by way of non-limiting example only, with reference to the accompanying drawings in which:

FIG. 1 is a schematic of a decellularization process for obtaining a dECM powder.

FIG. 2 shows a schematic layout of a decellularization reactor, reagents and biohazard waste.

FIG. 3 shows a workflow for a decellularization wash cycle.

FIG. 4 shows how pH at the start and end of the detergent wash cycles.

FIG. 5 is an extension of FIG. 4, showing how the initial detergent wash cycles end with lower pH, due to generation of cell debris and cell death caused by the decellularization process. It also shows the completion of the decellularization process indicated by the ˜same pH at the start and end of the wash (Wash 4)

FIG. 6 shows average pH of reagents against time in a reactor for clearing wash cycles, where the pH in the later cycles eventually starts to plateau close to DI water pH readings, indicating that the clearing process is nearing to an end and that the product is ready for downstream processes.

FIG. 7 shows % red and % blue taken from CIELAB values, a* and −b* respectively, of tissue samples for washes 0 to 11.

FIG. 8 shows % white taken from CIELAB values, L*, of tissue samples for washes 0 to 11.

FIG. 9 shows a schematic diagram of a tissue chamber assembly (left), with an image of decellularized tissue on a collection plate (right) which can be detached from the assembly.

FIG. 10 shows (left) dECM samples during a freeze-drying process and (right) the completely dried samples.

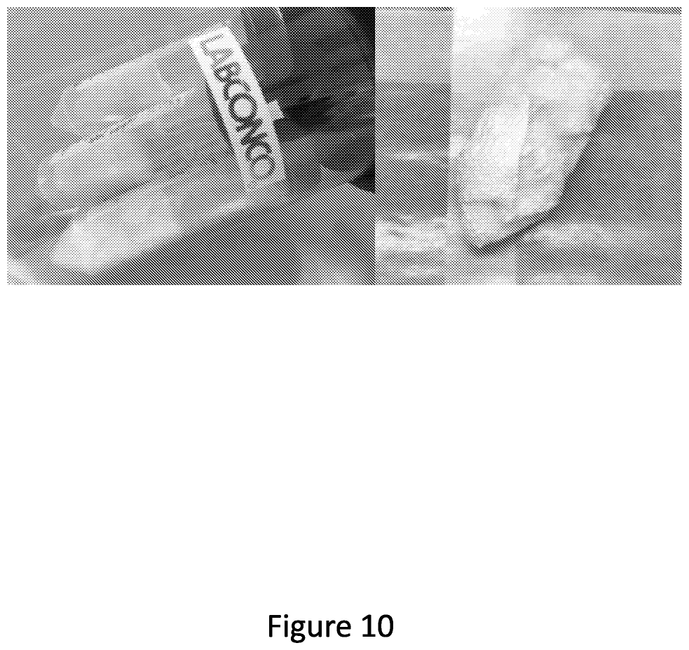

FIG. 11 shows cryomilled dECM powder, (left) top view, and (right) side view.

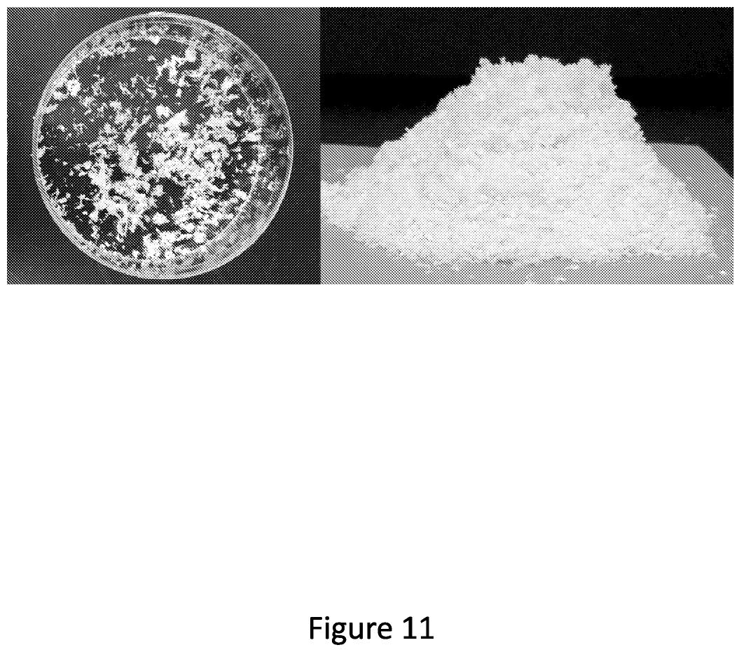

FIG. 12 shows images of tissue samples taken after washes 0, 4 and 11 (n=4).

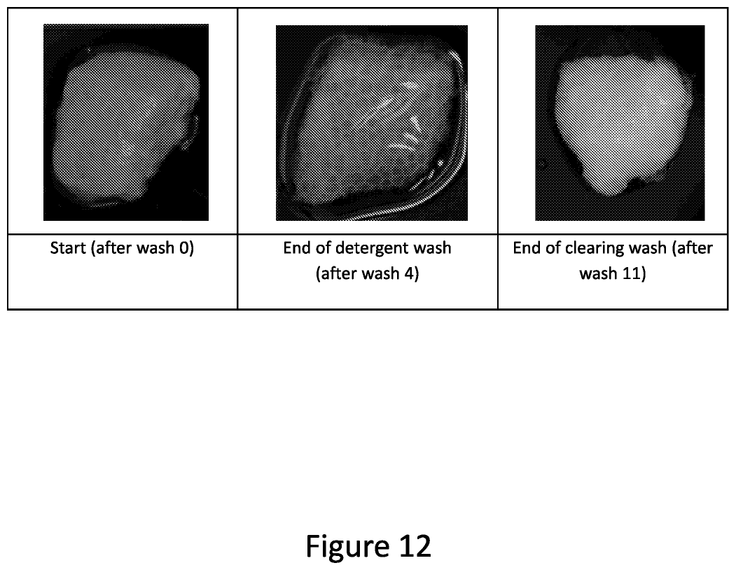

FIG. 13 shows a side perspective view of a decellularization reactor.

FIG. 14(A) to (D) show an assembly sequence of the decellularization reactor of FIG. 13.

FIG. 15 shows an exploded view of an example of a decellularization reactor.

FIG. 16 shows a partial exploded view of a decellularization reactor that shows an attachment mechanism of components of the decellularization reactor, and tissue chamber assembly.

FIG. 17 shows an exploded view of another example of a decellularization reactor, which can be adapted with various modules such as stirrers, types of agitation techniques etc. for decellularization of different samples.

FIG. 18 shows a reagent filling sequence for the decellularization reactor of FIG. 13.

FIG. 19 shows a cutaway view of the decellularization reactor of FIG. 13.

FIG. 20 shows a schematic of an automated decellularization system, connected to a pump which can pump in and out the reagents and wastes respectively.

FIG. 21 shows a side perspective view of a miniaturized decellularization reactor adapted for a heat plate and a temperature sensor.

### DETAILED DESCRIPTION

Embodiments relate to a standardized method of decellularization, and a decellularization device designed for a decellularization protocol, controlled by Adaptive Process Control capabilities to obtain decellularized extracellular matrix from small denominations of biological tissues. At least some embodiments may use non-destructive monitoring measurements such as pH or color (for example, CIELAB values) to track the progress of decellularization, while adaptively controlling parameters that influence the speed and extent of decellularization such as the intensity of agitation, the concentration of reagents or the number of wash cycles.

Small denominations of tissue samples may be consolidated within the decellularization reactor (dECM reactor). These small denominations can be in the form of cubes, cylinders, spheres, slices, discs, pellets or irregular shapes.

The decellularization method may follow a sequence of procedures that comprises agitation and decellularization, lyophilization, cryomilling, characterization and formation of biomaterial formulations, coatings, hydrogels or bioinks. The obtained dECM scaffold/biomaterial/structure/powder could be put through various physical and mechanical characterizations like scanning electron microscopy (SEM and SEM-EDX), Fourier Transform Infrared spectrometry (FTIR), thermogravimetric analysis (TGA), uniaxial/biaxial extension testing (Instron), texture and compression analysis (Texture profile analyser), contact angle measurement (via a goniometer) etc. The obtained dECM scaffold/biomaterial/structure/powder could be put through various biochemical characterizations to measure for specific ECM materials like collagen (hydroxyproline assay), sGAG (Dimethylmethylene Blue Assay), DNA (PicoGreen assay or Nanodrop), amino acids and sugars via high performance liquid chromatography (HPLC) and proteins and peptide analysis via Liquid chromatography-mass spectrometry (LC-MS) etc.

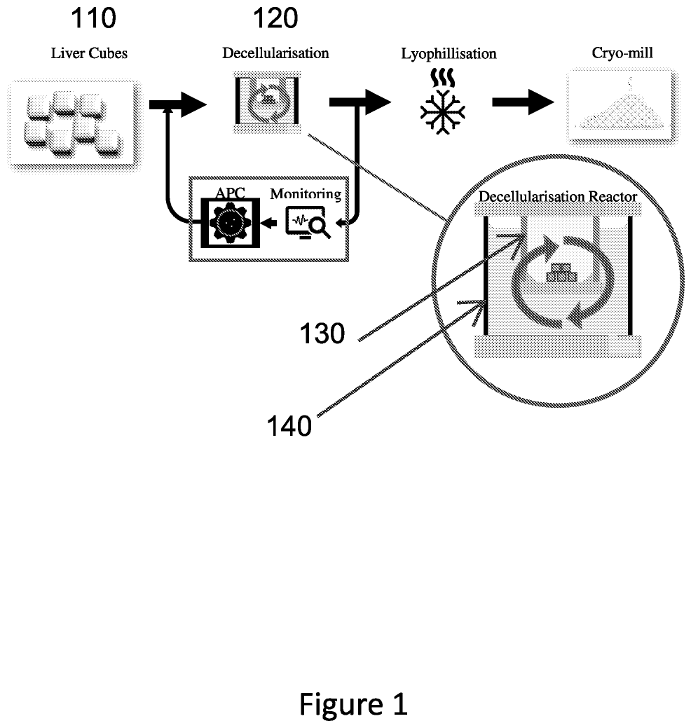

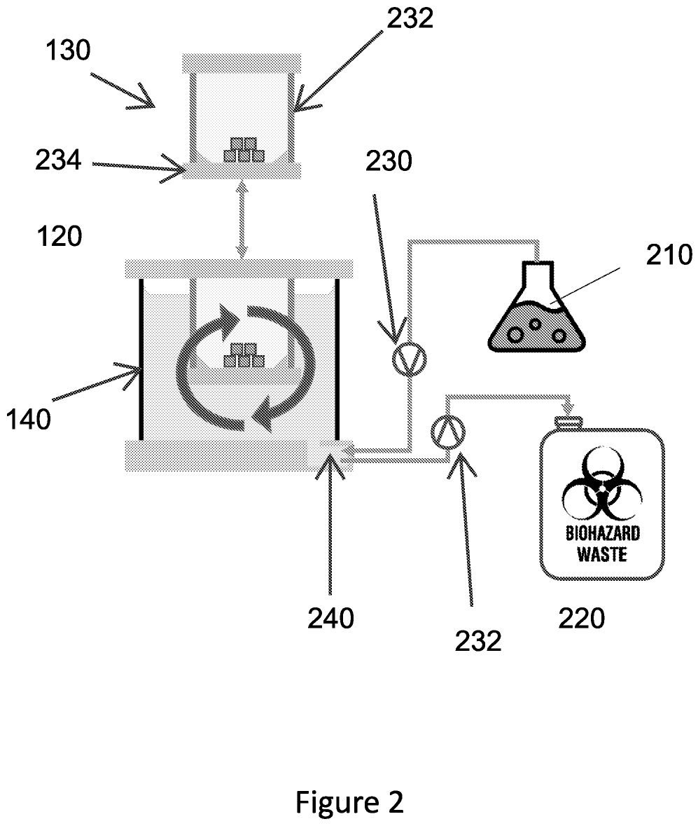

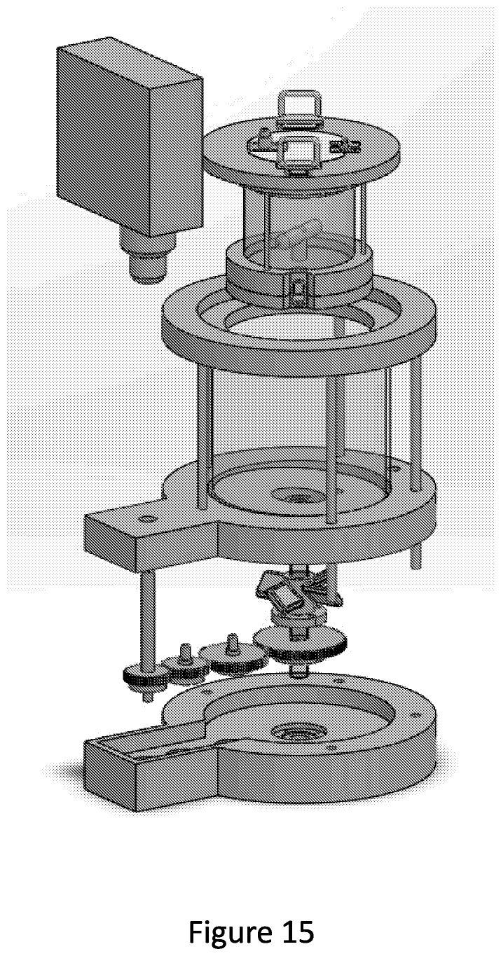

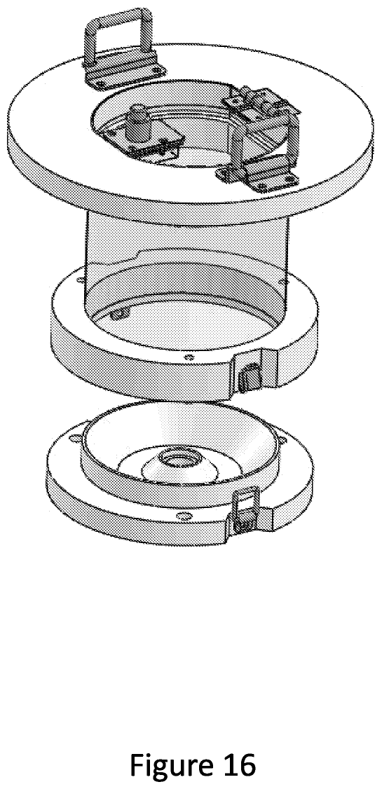

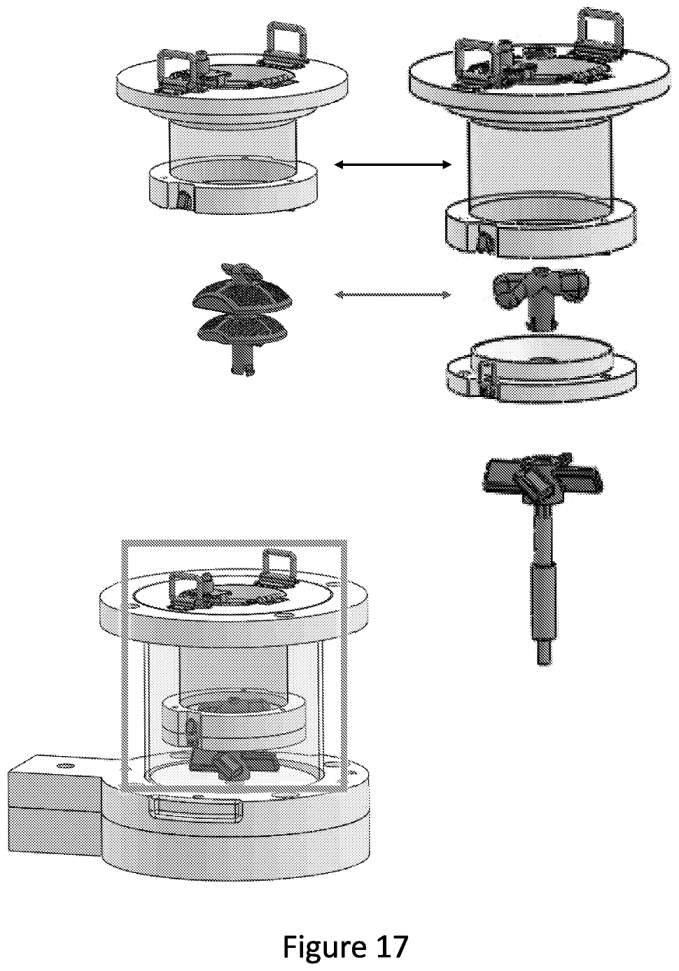

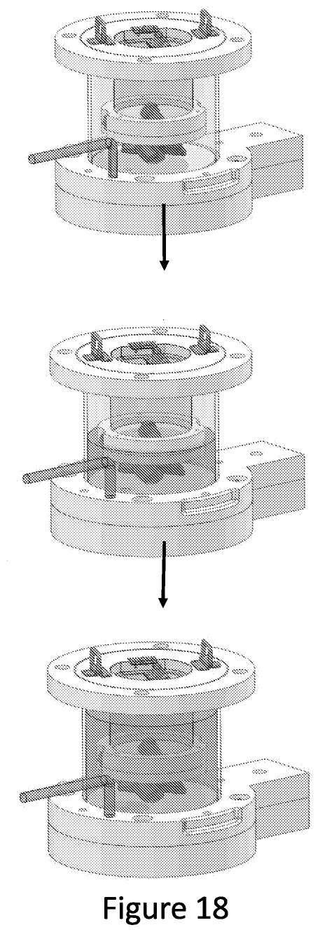

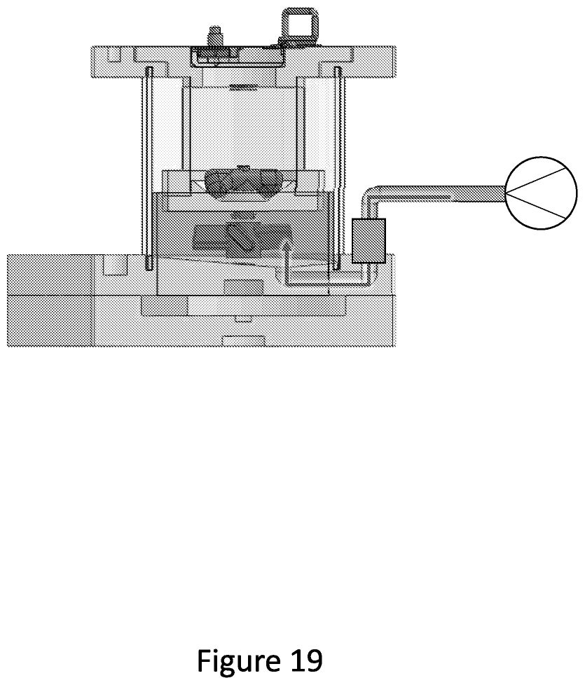

One or more of the parameters of the decellularization process (such as duration, concentration, viscosity and volume of reagents, intensity of agitation, temperature of the reagents) may be optimized to create a standardized method, and a specialized decellularization reactor may be designed to consolidate the steps into a single piece of equipment, examples of which are shown in FIG. 1, FIG. 2 and FIGS. 13-20. The components in the reactor are designed to streamline the decellularization process, while minimizing the required human interaction.

Furthermore, non-destructive monitoring capabilities may be included to track the progress of decellularization, while the Adaptive Process Control (a system that automatically adjusts itself to have a performance which is optimum to some preassigned criteria) may adjust the parameters to optimize the decellularization process where needed.

Although reducing the animal organ into tissues destroys the structure of the organs, the structure can be eventually rebuilt into tissue constructs using 3D printing. In some embodiments, as described in the following, a decellularization technique focuses on using smaller denominations of animal tissue to increase efficiency and production. The porcine liver was used as a proof of concept in these experiments because of the numerous advantages it has over organs from non-human primates. Due to its high breeding potential, it provides a plentiful and sustainable source for the decellularization process. This is essential, especially for organ-derived bioink, making porcine sources a useful candidate for bioink formulation. Alternative proof of concept experiments were also performed using fungi, plant, human skin, animal skin, algae tissue samples.

Some embodiments relate to a device that is specially designed to consolidate and conduct these processes, under optimal conditions and parameters; a Decellularization Reactor with the potential to scale-up and employ automation for automated manufacturing. Monitoring techniques are also used to track the progress of decellularization and feedback the progress into an Adaptive Process Control. These feedback mechanisms control the progress of decellularization by responding to the measured extent of decellularization of the tissue samples, by adjusting the control parameters. Thereby, adapting the standardized decellularization method to different sample types between different species, or different sample (tissue) types within the same species.

Adjusting the control parameters of the decellularization process includes adjusting an amount and/or a concentration of the reagent, or of a component thereof. Alternatively, adjusting may include adjusting a speed and/or a duration of said agitating. In some embodiments, adjusting may comprise adjusting the number of wash cycles. One or more control parameters may be adjusted by the embodiments to optimize the decellularization process.

The reactor vessel may comprise a tissue chamber for containing the sample, and a reactor chamber within which the tissue chamber is removably seated. The reactor may comprise an agitation or circulation system which can be in the form of an impeller, in line centrally with the inner chamber. This system enables continuous circulation and exchange of reagents and waste between the tissue and reactor chamber. The reactor chamber may also comprise a port which acts as both inlet and outlet for admitting fresh reagents and removing used reagents respectively, through a bottom-up delivery method.

The tissue chamber may be detachable from the rest of the body of the reactor. The tissue chamber may comprise sample containment and extraction systems allowing convenient movement of the tissue chamber while allowing the extraction of the chamber contents when necessary.

The surrounding wall of the reactor chamber may be made of a clear material, such as clear acrylic or polypropylene or polycarbonate or glass, which enables optical based sensors to be attached externally to monitor the progress of the decellularization either directly based on the colour/composition of the wash reagents or tissue samples, or by attachments on the inner side of the outer chamber clear walls, inside the reagent which undergoes changes in composition, hence enabling optical-based probing for monitoring of pH/dO₂/etc. The supporting elements and mechanical components of the reactor could be fabricated using stainless steel such as SUS304 stainless steel material.

A filter element (perforated sheet) may form the walls on the tissue chamber. Perforations of the filter element may be sized to enable both ingress and egress of fresh wash reagents and waste cellular materials, while containing the bulk of the target tissue samples within the tissue chamber. This minimizes sample loss, while enabling continuous and consistent decellularization with the impeller within the perforated walls, and clean sample collection within the tissue chamber.

The tissue chamber may also comprise an agitation or circulation system, which may be in connection with the agitation or circulation system in the reactor chamber or can be independent of that. This can be in the form of an impeller design depending on the type of samples. The system enables continuous and homogeneous decellularization. The design and speed for the system may vary for different samples to minimize destruction of the sample while optimizing sample yield.

The tissue chamber may have a removable base (collection plate) with a well for harvesting the decellularized tissue sample. While the base is detached from the upper part, the samples can be easily collected for further processing. The tissue chamber may also have a lid that can be clear or opaque depending on the type of sensors. The lid enables input of samples in a clean and organized fashion. Clear lids made of acrylic or polypropylene or polycarbonate or glass can be used to monitor the samples throughout the wash, through optical methods, based on the colour and/or composition of the wash reagents or tissue samples. The lid also enables tissue extraction during washes for further analysis.

### Initial Characterization of Sample

The decellularization reactor may have modular components to adapt the techniques to a wide range of different tissue samples. These tissue samples can derive from organisms across different Kingdom, Class, to Family (including but not limited to humans, animals, plants, algae and fungi), and from different organs and tissues (including but not limited to skin, liver, heart, kidney, lungs, stomach, intestines, bladder, blood vessels, from animals OR stem, leaves, root, fruits, flowers, buds from plants OR stalk, hyphae, cap, fruiting body of fungi OR stipe (cortex and medulla), blade, holdfast of algae/microalgae). The tissues could also have been artificially (in-vitro culture) made in the lab. These artificially made tissues could be in the form of cell spheroids, tissue spheroids, cell sheets, tissues on microbeads, tissues on microcarriers, tissues on scaffolds, tissues on hydrogels, tissues on substrates.

An initial characterization of the tissue sample may be used to determine the appropriate process for decellularization and establish a starting condition or baseline for future Adaptive Process Control. Characteristics may include:
The Kingdom, Class and/or Family of the organism the sample is taken from,
The organ and tissue the sample is taken from,
The sample shape and size, and
Physical characteristics such as tissue density, fat content, protein content, glucose levels, rigidity, etc.

One set of initial characteristic input can be:
Sus Scrofa pig, approximately 75 kg
Liver tissue taken from the right medial lobe
12 mm×12 mm×12 mm cubes
Average measured fat content of 6.5%

Alternative characteristic inputs to determine the appropriate process for decellularization and establish a starting condition or baseline for future Adaptive Process Control include fungi, plant, human skin, animal skin, algae tissue samples.

Adaptive Process Control uses the input values and measurements obtained from monitoring processes throughout the decellularization process to optimize and adjust the control parameters. Some control parameters that can be used for Adaptive Process Control include the number of washes, the concentration of reagents, the duration of each wash and the intensity of agitation.

After identifying the characteristics of the tissue sample, a DECM reactor of a suitable scale can be used to efficiently decellularize and optimize the reagent usage. For example, in one embodiment of a larger DECM reactor can be used for 200 g-400 g tissue samples. In a second embodiment, a smaller DECM reactor can be used for 50 g-100 g tissue samples. The tissue samples may include porcine liver cubes.

### Design of the Decellularization Reactor

A decellularization reactor may be designed specially to suit the decellularization processes or adapted with modules specific to the tissue sample.

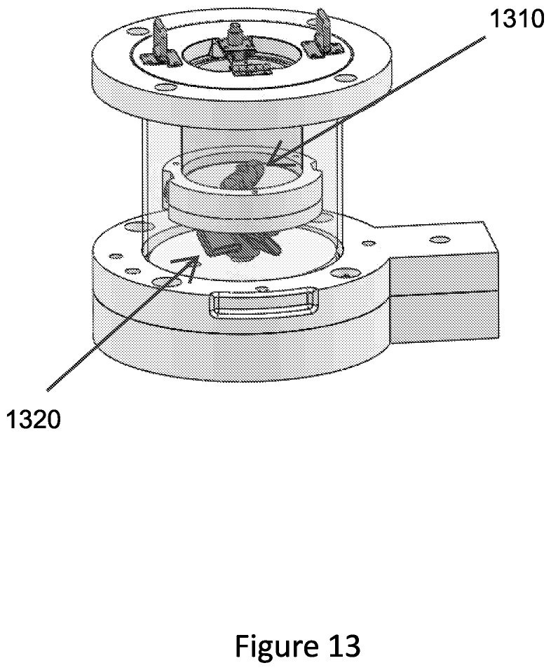

The loading of tissue samples, reagent, decellularization and monitoring of progress occurs within one reactor, which minimizes the number of components and required movement of equipment. Additionally, circulation can occur between dual chambers, accelerating the decellularization process, which can also be monitored by a colorimeter or camera. These allow the progress to be tracked and Adaptive Process Control can be used to adjust the processes where necessary to optimize the decellularization. Example designs of decellularization reactors are further elaborated in FIGS. 13 to 21.

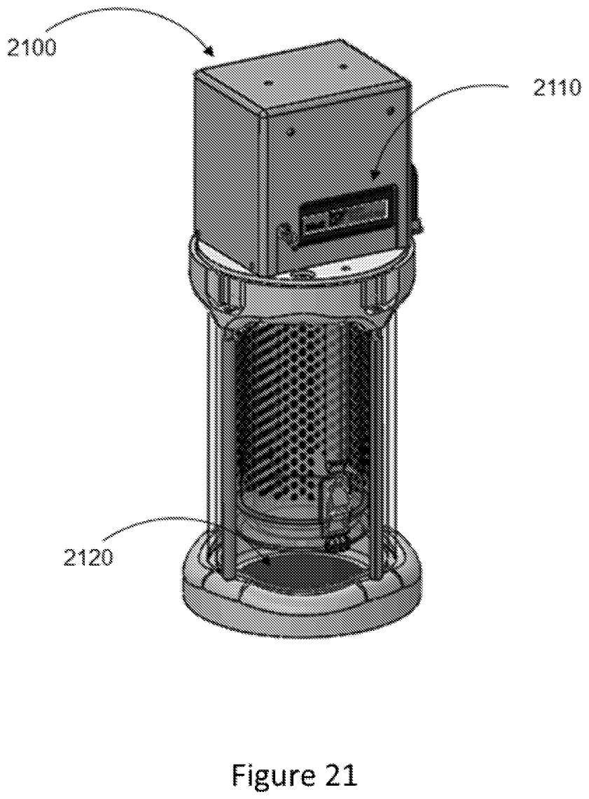

The reactor of some embodiments has the approximate dimensions of 295 mm (H)×300 mm (W)×400 mm (L). The reactor of some embodiments decellularizes samples between the masses of 100 g to 1500 g or alternatively 200 g to 400 g or alternatively between 25 g to 100 g. The reactor of some embodiments may have a smaller miniaturized size with a volume of approximately 1.6 L. Such reactors may handle a sample of mass approximately between 50 g to 100 g. The miniaturized reactor may have a size of approximately 15 cm×15 cm×35 cm. FIG. 21 illustrates one such miniaturized reactor 2100. The reactor 2100 also has a temperature sensor provided in either or both the inner and outer chambers of the reactor. An LCD display 2110 is provided to display the temperature of the sample inside the reactor. Also provided in reactor 2100 is a metal plate 2120 which can improve conduction of heat from a source of heat such as a heat plate or a heating element. The source of heat may or may not be an integral part of the reactor 2100. The reactor of some embodiments may include circuitry that implements a feedback loop between the temperature sensors and a source of heat conveying heat to the metal plate 2120 to manage the temperature of samples inside the reactor.

The size/capacity of the reactors may be varied to accommodate the specific requirements of the decellularization framework or research environment.

FIG. 2 illustrates a schematic layout of a decellularization reactor 120, reagent 210 and biohazard waste 220. Pumps 230 and 232 are provided to facilitate the movement of the reagent and biohazard waste in and out of the reactor chamber 140 through the reagent exchange port 240. The port is located below the tissue chamber.

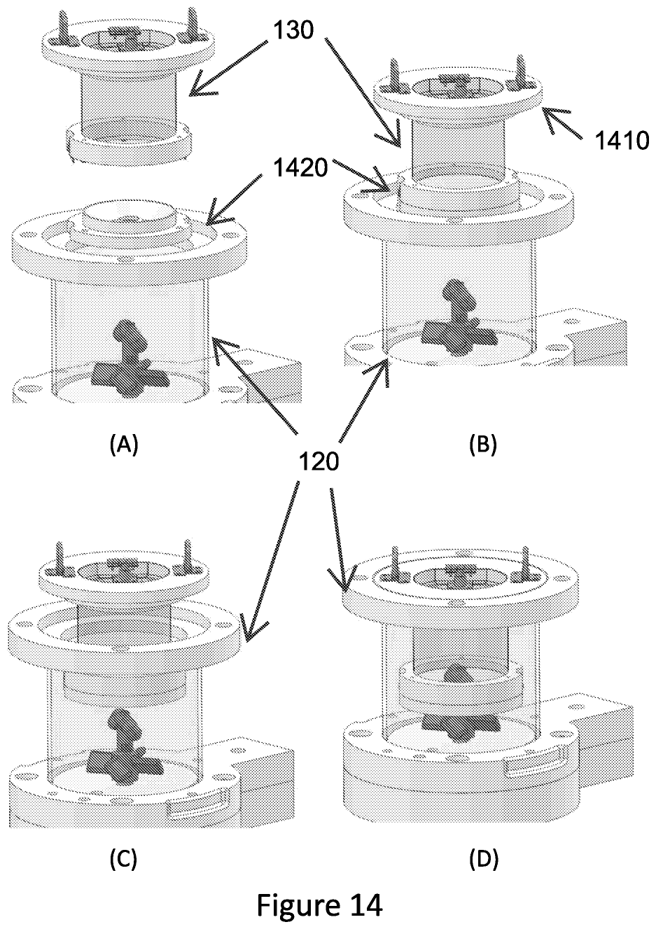

A tissue chamber 130 of some reactors may comprise a cylindrical sidewall. In some embodiments, the tissue chamber is detachable from the reactor chamber. After detachment, the tissue chamber may be handled independently of the rest of the reactor. FIG. 14 illustrates a series of images wherein the tissue chamber is progressively assembled within a reactor. The tissue chamber comprises a flange 1410 at an upper end thereof. The flange is adapted to mate with a recessed seat 1420 at an upper end of the reactor chamber.

The agitation system of the reactor may comprise one impeller. In some embodiments, the agitation system may comprise a first impeller in the tissue chamber, and a second impeller in the reactor chamber. The first and second impeller may be mounted to a common shaft. FIG. 13 illustrates one such reactor with a first impeller 1310 and a second impeller 1320. The first impeller and the second impeller may be independently movable. In some embodiments, the tissue chamber is rotatable such that the agitation system comprises the tissue chamber.

The agitation system may comprise a vortex generator. The vortex generator comprises a submerged pump, a tangential facing nozzle inside the tissue chamber, and/or a central draining hole in the reactor chamber. The vortex generator may also comprise means for generating air or liquid jets.

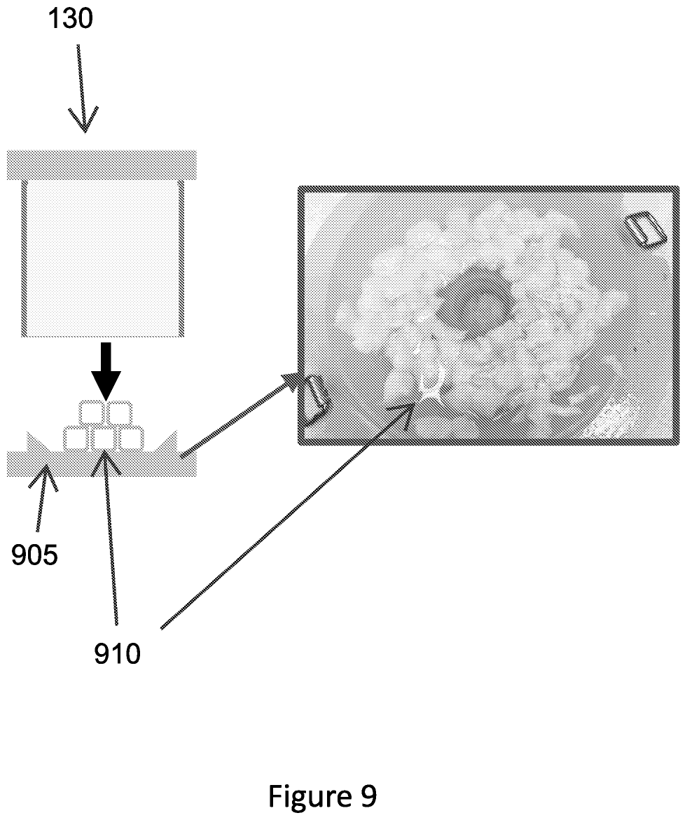

The tissue chamber may comprise a detachable base. FIG. 9 illustrates a detachable base 910 of the tissue chamber. The base may have a concave or a recessed collection surface. Further, a lid of the tissue chamber and/or the sidewall of the tissue chamber may be transparent or translucent to allow visual inspection of the contents of the tissue chamber. In some embodiments, a sidewall of the reactor chamber may be transparent or translucent.

### Decellularization System

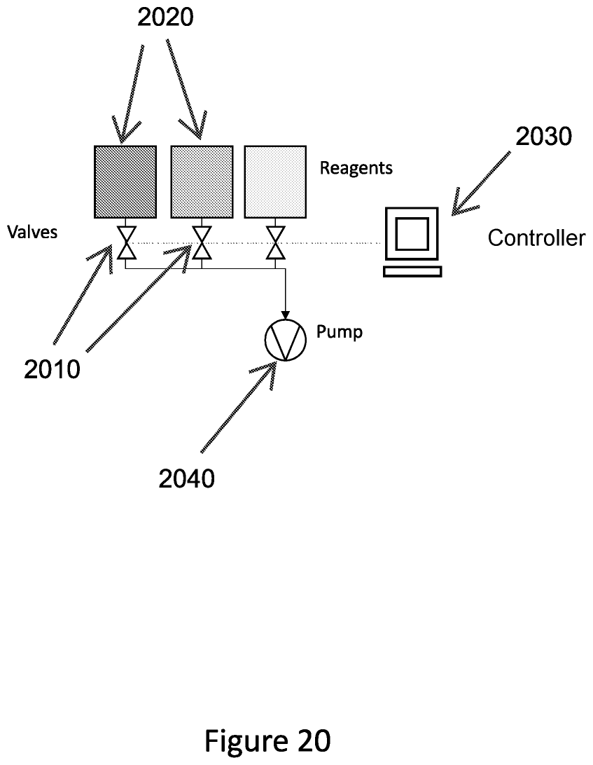

Some embodiments are directed to a decellularization system. The system may include a decellularization reactor, a reagent delivery system connectable to the port of the decellularization reactor; and a controller that is operably connectable to the reagent delivery system and the agitation system of the reactor. FIG. 20 illustrates a part of the decellularization system with a reagent delivery system comprising a plurality of reagents 2020 that pass through a respective valve 2010 to the pump 2040. The pump 2040 is connected to the port of the decellularization reactor to which the reagents are delivered.

A controller 2030 controls the operation of the values 2010 to regulate the supply of the reagents to the pump to thereby control the concentration and/or amount of reagents in the decellularization reactor. The controller is also configured to cause the decellularization reactor to perform one or more wash cycles each comprising: at least partially filling, by the reagent delivery system, the reactor chamber with reagent such that the tissue sample is at least partially submerged in the reagent; and agitating, by the agitation system, the tissue sample and/or the reagent such that the tissue sample circulates within the reagent, while retaining the tissue sample in the tissue chamber.

The decellularization system may comprise sensors to measure parameters relevant to the decellularization process. The sensors provide data to the controller that may control the decellularization process based on the data received from the sensors and the adaptive control logic embedded in the controller. The sensors may monitor: pH of the reagent, optical properties of the reagent and/or the tissue sample, images of the tissue sample and/or the reagent. The sensor may include optical sensors that monitor optical properties during decellularization. Optical properties may include spectroscopic properties of regions of the one or more images containing the tissue sample and/or spectroscopic properties of regions of the one or more images containing the reagent. Optical properties may include colorimetric properties of regions of the one or more images containing the tissue sample and/or colorimetric properties of regions of the one or more images containing the reagent. Sensors may monitor turbidity and/or temperature of the reagent in the reactor chamber. Sensors may also monitor the mass of the tissues at one or more stages of the decellularization process. Sensors may include optoelectronic meters, mass balance or temperature probes etc.

### Decellularization Process

FIG. 1 illustrates a schematic of a decellularization process. Liver cubes 110 serve as a starting sample for the decelluralization process performed in the reactor 120 wherein the adaptive process control and monitoring operations are performed. The reactor 120 comprises a tissue chamber 130 that is in fluid communication with the reactor chamber 140. Liver cubes/tissue samples are placed in the tissue chamber 130 (also referred to as an inner chamber). The decelluralized output of the tissue chamber is further subjected to lyophilization and cryomilling to obtain the dECM powder.

The tissue chamber is in fluid communication with the reactor chamber. The tissue chamber is nested within the reactor chamber. The tissue chamber comprising a base 234 and one or more sidewalls 232 extending from the base. At least one of said one or more sidewalls comprises a plurality of apertures for passage of fluid between the tissue chamber and the reactor chamber while retaining the tissue sample within the tissue chamber.

After identifying the characteristics of the tissue sample, a DECM reactor of the appropriate scale can be used to efficiently decellularize and optimize the reagent usage, e.g. One embodiment of a larger DECM reactor can be used for 200 g-400 g of porcine liver cubes, or a miniaturized DECM reactor for tissue samples with a mass in the range of 50 g-100 g.

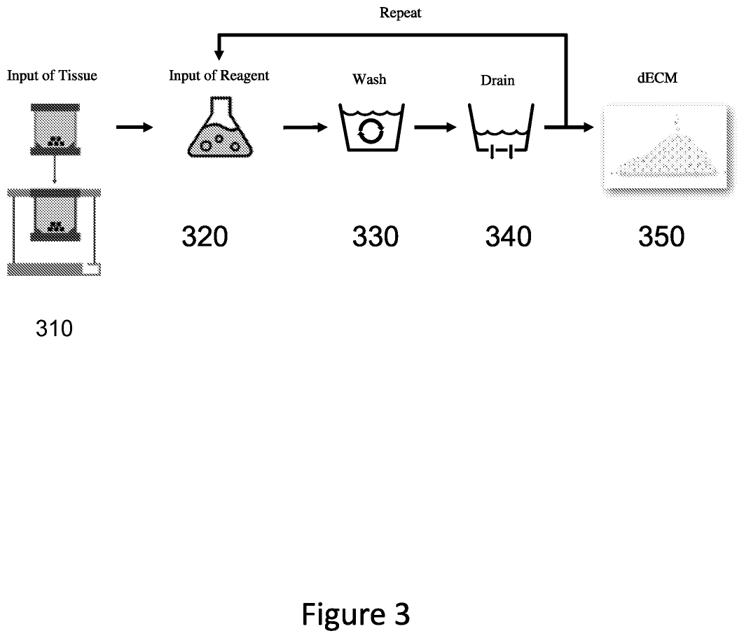

FIG. 3 illustrates a workflow for a decellularization wash cycle using liver tissue samples as an illustrative input to the process at step 310.

### Loading of Liver

With the fat percentage of the sample obtained, the respective process is selected. An optimal mass of approximately 200-400 g of liver cubes is used in each wash cycle, allowing efficient usage of the reactor as well as to prevent insufficient digestion of the samples. Overcrowding of samples will result in an increased contact and abrasion, which increase the chances of tearing and shredding. Additionally, the speed and movement of samples could be blocked by physical interference of other samples, lowering the circulation, and slowing the decellularization.

### Input & Draining of Reagents

After loading the liver tissue at step 310, a cycle of loading reagents 320, washing 330, and draining of reagents 340 is performed within the reactor to obtain dECM 350.

The reagents may be input by connecting rubber tubing through a peristaltic pump, with the tail of the rubber tubing submerged in the reservoir containing 4.5 L of reagent. The reagent may fill the reactor from the bottom up. Filling from the bottom up allows a gentle input of reagents into the reactor to prevent tearing of liver cubes due to splashes or impact from fast falling reagents.

Draining of the reagents may be carried out by placing the tail of the rubber tubing in a bio-waste carboy that is at a lower height. The used reagents can be drained out using gravity. A single inlet-outlet point is used for easier control while keeping the reactor compact. Quick connects are used to facilitate cleaning and mobility of equipment.

### Wash Cycle

A pre-wash may be performed with DI water to remove as much of the animal blood as possible; blood clots can interfere with the decellularization process. This also ensures sufficient visibility in the subsequent decellularization washes, for visual inspection to monitor the decellularization process.

Two sets of washes may be done, with several repetitions of the respective reagents to fully decellularize the liver cubes:
(a) detergent washes: where detergents are used to break down the DNA of cells such as Triton X in NH₄OH, which will be removed as waste, and
(b) clearing washes: where ionic buffer solutions such as PBS and DI water are used to remove the detergents from the dECM

The number of wash cycles, durations of wash cycles and concentration of reagents are parameters that may be controlled by the Adaptive Process to ensure optimum yield.

Throughout the wash cycles, the reagent and liver cubes are kept in a continuous circulation to facilitate digestion and exchange of reagents and cellular materials between the dual chambers. This circulation can be sustained using impellers driven by a motor or water jets to create a vortex. Additionally, the revolution provides a centrifugal force on the liver cubes, from inside the tissue chamber, against the perforated sheet. The holes of the perforated sheet are only large enough for the cellular materials to exit and thus, separate the cellular materials from the liver cubes through the washes. These holes of the perforated sheet may have a wider range depending on the tissue samples such as from plant, fungi, or algae.

The RPM may also be controlled as an Adaptive Process because sufficient velocity is needed to generate lift on the liver cubes and sustain a continuous flow, but below the threshold for tearing and shredding the liver tissue.

A wash cycle change may be performed when a change in at least one parameter of the reagent is less than a threshold, or when a change in at least one parameter of the tissue Sample is Less than a Threshold, or when at Least One Parameter of the Reagent Reaches a threshold, or when at least one parameter of the tissue sample reaches a threshold.

### Monitoring the Progress of Decellularization

### pH Measurement

Monitoring capabilities of the decellularization reactor can include sensors integrated into the tissue chamber and/or reactor chamber. Said monitoring may comprise at-line measurement of the pH of the reagent using a sensor. The sensors can measure pH, dO₂, metabolite (e.g. lactate, lactic acid). One embodiment can measure the pH of the reagents in the reactor chamber, and input it into the Adaptive Process Control to determine the progress of decellularization in respect to the current wash cycle, and adapt the parameters where needed.

TABLE 1
Examples of pH and Wash number for reagent
solutions, before adding into the reactor

| Reagent | Wash no. | pH |
|---|---|---|
| 2% TritonX-100 and 0.4% NH₄OH | 1-4 | 10.7 |
| 1X PBS | 5-6 | 7.5 |
| DI H₂O | 7-9, 11 | 5.5-7.0 |
| 4% Ethanol | 10 | 5.8 |

Table 1 shows some examples of the reagents and the pH, and the wash cycle when they are added. pH measurement provides a good measurement of the decellularization detergent and clearing wash progress. In the detergent washes, reagents were used to remove the cells from the ECM, thus resulting in cell death and debris which contribute to increased release of acidic reagents, such as metabolites and lactic acid. In clearing washes, the aim was to clean the samples as much as possible of the reagents used in the detergent washes, which may be toxic for downstream applications, while restoring a neutral pH.

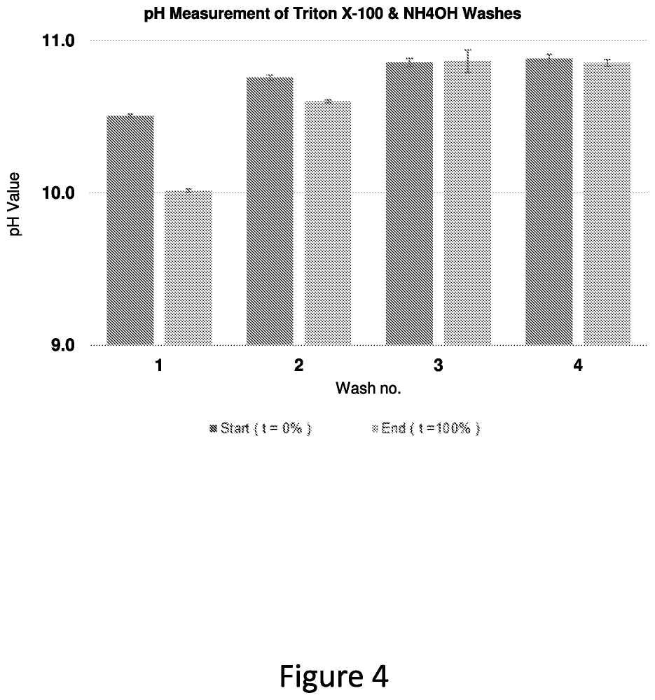

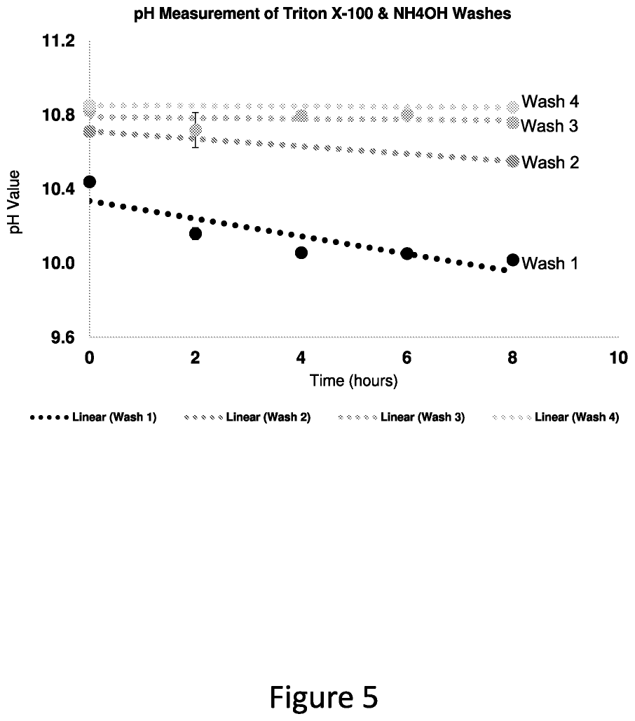

pH can hence be used to indicate this progress of decellularization, while ascertaining the end point of decellularization, and when to initiate clearing wash cycles. FIG. 4 shows the initial and final pH vs the wash number for each detergent wash step. FIG. 5 is an extension of FIG. 4, which shows the individual change in pH for each wash. It can be seen from both figures, that with multiple rounds of the detergent washes, less cellular material remained for decellularization, which results in a more consistent pH at the start and end.

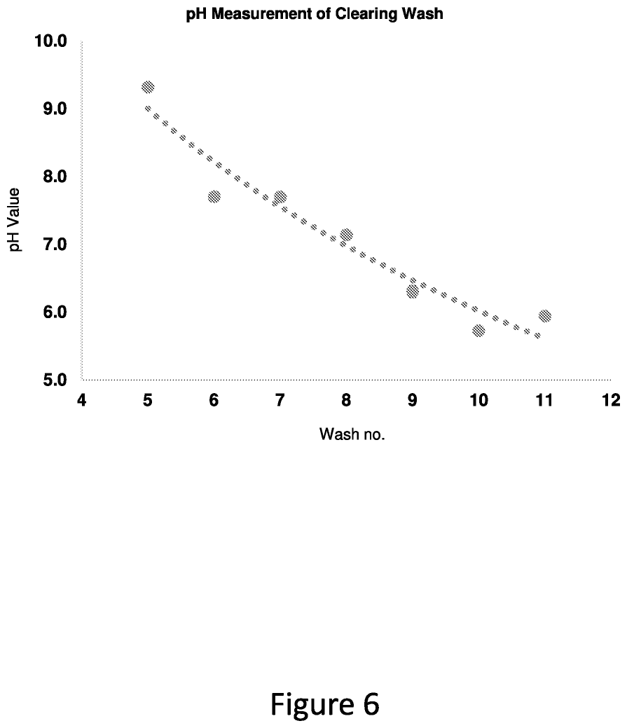

FIG. 6 illustrates the pH measurement curve for the respective clearance wash cycles. It can be seen that pH can be used to clearly track the removal of the toxic detergent wash reagents as the curve subsequently reaches the pH values of DI water after multiple washes, indicating that mostly DI water remains.

### CIELAB Measurement

Throughout the decellularization process, a visual inspection of the tissue samples may be performed using a camera to evaluate the integrity of the tissue samples (analyzing the degree of shredding of samples). Additionally, the opacity and color of the tissue samples between washes will be recorded to track the progress of decellularization.

Said monitoring may comprise monitoring one or more optical properties of the reagent and/or the tissue sample by capturing one or more images. The one or more optical properties comprise colorimetric properties of regions of the one or more images containing the tissue sample and/or colorimetric properties of regions of the one or more images containing the reagent. Alternatively, the one or more optical properties may comprise CIELAB values. Alternatively, the one or more optical properties comprise spectroscopic properties of regions of the one or more images containing the tissue sample and/or spectroscopic properties of regions of the one or more images containing the reagent.

In one embodiment, the camera captures images using the same settings and in a controlled environment; overhead and ambient lighting. These images can be broken down to obtain the CIELAB values (L* for the lightness from black (0) to white (100), a* from green (−) to red (+), and b* from blue (−) to yellow (+)), which could reveal the extent of decellularization and the extent which the detergents are cleared from the tissue samples.

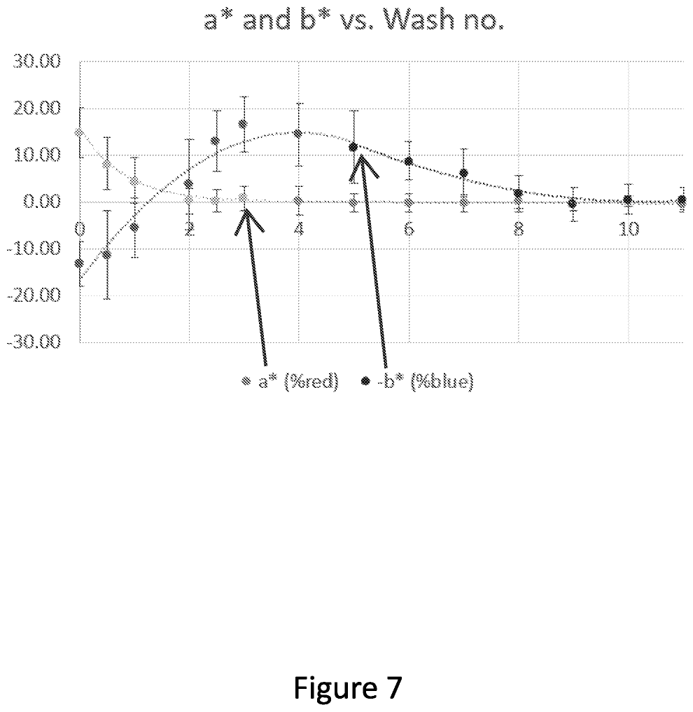

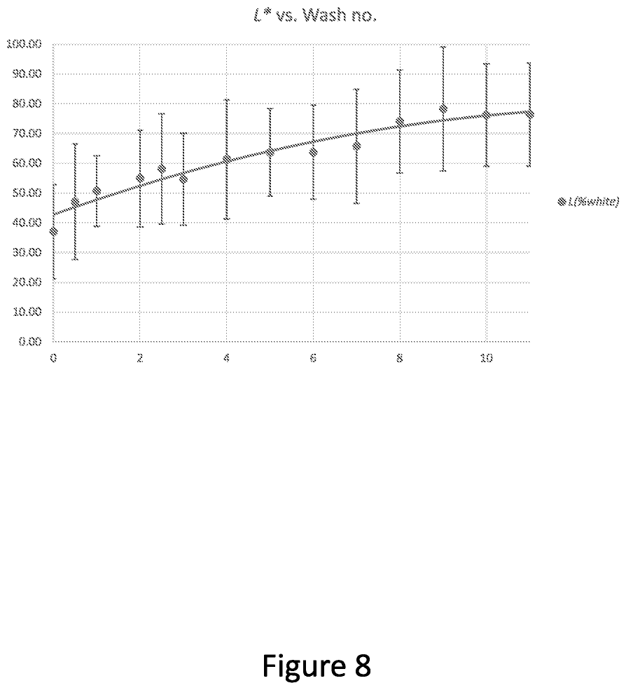

Generally, tissue samples decolourise to white and lose their transparency through the decellularisation process, while regaining a milky white opacity as the detergents are cleared. The CIELAB values reveal this trend of decolourisation and maps it with the trends observed for the tissue sample to ascertain the extent of decellularisation and detergent clearing, as seen in FIGS. 7 and 8.

Collection of dECM and Maintenance of Reactor

After the final wash and draining of reagents, the base of the tissue chamber can be disconnected to reveal a Collection Plate 905, which holds all the dECM samples 910 as illustrated in FIG. 9.

The decellularization chambers of the tissue chamber and reactor chamber can be easily rinsed with water and wiped down with ethanol, to allow little downtime between decellularization batches. Smaller components and diassembled parts of the tissue chamber and reactor chambers can be put through the autoclave.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method for decellularizing a tissue sample to produce decellularized extracellular matrix (dECM) material, the method comprising:
placing the tissue sample in a tissue chamber that is in fluid communication with a reactor chamber;
performing one or more wash cycles each comprising: at least partially filling the reactor chamber with a reagent such that the tissue sample is at least partially submerged in the reagent; and agitating the tissue sample and/or the reagent such that the tissue sample circulates in the reagent, while retaining the tissue sample in the tissue chamber;
monitoring at least one parameter of the reagent and/or at least one parameter of the tissue sample, by monitoring one or more optical properties of the reagent and/or the tissue sample; and
adjusting one or more wash cycles based on said monitoring.

2. A method according to claim 1, wherein said monitoring comprises monitoring the pH of the reagent.

3. A method according to claim 2, wherein said monitoring comprises at-line measurement of the pH of the reagent.

4. (canceled)

5. A method according to claim 1, wherein the one or more optical properties comprise spectroscopic properties of regions of the one or more images containing the tissue sample and/or spectroscopic properties of regions of the one or more images containing the reagent.

6. A method according to claim 1, wherein said adjusting occurs within a wash cycle of the one or more wash cycles.

7. A method according to claim 1, wherein the method comprises two or more wash cycles and said adjusting occurs between wash cycles.

8. A method according to claim 1, wherein the reactor chamber is at least partially filled from below the tissue sample.

9. A method according to claim 1, further comprising determining at least one initial characteristic of the tissue sample prior to placing the tissue sample in the tissue chamber.

10. A method according to claim 1, wherein said adjusting comprises adjusting an amount and/or a concentration of the reagent, or of a component thereof.

11. A method according to claim 1, wherein said adjusting comprises adjusting a speed and/or a duration of said agitating.

12. A method according to claim 1, wherein said adjusting comprises adjusting the number of wash cycles.

13. A method according to claim 1, wherein each said wash cycle comprises, after said agitating, draining the reagent from the reactor chamber, while retaining the tissue sample in the tissue chamber.

14. A method according to claim 1, further comprising performing at least one clearing wash cycle for removing residual reagent from the dECM material.

15. A method according to claim 1, wherein a wash cycle change is performed when a change in at least one parameter of the reagent is less than a threshold; or when a change in at least one parameter of the tissue sample is less than a threshold; or when at least one parameter of the reagent reaches a threshold; or when at least one parameter of the tissue sample reaches a threshold.

16-32. (canceled)

33. A decellularization system comprising:
a decellularization reactor for decellularizing a tissue sample to produce decellularized extracellular matrix (dECM) material, the decellularization reactor comprising:
a reactor chamber;
a tissue chamber that is in fluid communication with the reactor chamber, the tissue chamber comprising a base and one or more sidewalls extending from the base, at least one of said one or more sidewalls comprising a plurality of apertures for passage of fluid between the tissue chamber and the reactor chamber while retaining the tissue sample within the tissue chamber;
a port in fluid communication with the reactor chamber and the tissue chamber for introducing reagent into the chambers in use; and
an agitation system for agitating, in use, the reagent and/or the tissue sample such that the tissue sample circulates in the reagent in the tissue chamber;
a reagent delivery system connectable to the port of the decellularization reactor;
a controller that is operably connectable to the reagent delivery system and the agitation system, the controller being configured to cause the decellularization reactor to perform one or more wash cycles each comprising: at least partially filling, by the reagent delivery system, the reactor chamber with reagent such that the tissue sample is at least partially submerged in the reagent; and agitating, by the agitation system, the tissue sample and/or the reagent such that the tissue sample circulates within the reagent, while retaining the tissue sample in the tissue chamber; and
one or more sensors for measuring at least one parameter of the reagent and/or at least one parameter of the tissue sample, wherein the one or more sensors comprise one or more optical sensors for measuring one or more optical properties of the reagent and/or the tissue sample.

34. (canceled)

35. A decellularization system according to claim 33, wherein the controller is configured to: monitor at least one parameter of the reagent and/or at least one parameter of the tissue sample; and adjust the one or more wash cycles based on said monitoring.

36. A decellularization system according to claim 35, wherein the controller is configured to monitor the pH of the reagent.

37-40. (canceled)

41. A decellularization system according to claim 33, wherein the controller is configured to adjust an amount and/or a concentration of the reagent, or of a component thereof.

42. (canceled)

43. A decellularization system according to claim 33, wherein said adjusting comprises adjusting the number of wash cycles.

44. A decellularization system according to claim 33, wherein each said wash cycle comprises, after said agitating, draining the reagent from the reactor chamber, while retaining the tissue sample in the tissue chamber.

45. (canceled)

46. A decellularization system according to claim 33, wherein the controller is configured to:
detect when:
the change in the at least one parameter of the reagent; or the change in the at least one parameter of the tissue sample is less than a threshold; or
the at least one parameter of the reagent reaches a threshold; or
the at least one parameter of the tissue sample reaches a threshold; and
responsive to the detection, cause the decellularization reactor to perform at least one wash cycle change.

47-50. (canceled)
